(19) United States
(12) Patent Application Publication
(10) Pub. No.: US 2025/0064326 A1
(43) Pub. Date: Feb. 27, 2025

(54) Wearable Device for Detecting a Cardiac Arrest

(71) Applicant: Wear It System Holding S.A., Plan- les- Ouates (CH)

(72) Inventors: Iddo Johanan Gideon Hanse, Plan- les- Ouates (CH); Kambiz Ebrahimkheil, Plan- les- Ouates (CH)

(21) Appl. No.: 18/810,003

(22) Filed: Aug. 20, 2024

(30) Foreign Application Priority Data
Aug. 23, 2023 (NL) 2035659

(51) Int. Cl. A61B 5/0205; A61B 5/00; A61B 5/021; A61B 5/024; A61B 5/11

(52) U.S. Cl. CPC A61B 5/0205; A61B 5/1112; A61B 5/6802; A61B 5/6844; A61B 5/7225; A61B 5/746; A61B 5/02116; A61B 5/02416; A61B 2562/0219

(57) ABSTRACT

A wearable device that uses single-site measured PPG signals in order to detect potential cardiac arrests, by detecting a loss in the amplitude of the PPG signal in combination with the morphology of the PPG signal and fall and wearing detection steps.

### CROSS-REFERENCE TO RELATED APPLICATIONS

This application claims priority to and the benefit of Netherlands Patent Application No. 2035659, entitled “WEARABLE DEVICE FOR DETECTING A CARDIAC ARREST”, filed Aug. 23, 2023, and the specifications and claims thereof are incorporated herein by reference.

### BACKGROUND OF THE INVENTION

### Field of the Invention

The invention relates to a wearable device for non-invasively detecting a cardiac arrest of a wearer, using multi-wavelength photoplethysmography (PPG) signals obtained by said wearable device from peripheral blood vessels. This approach holds potential in enabling early detection of cardiac arrest, which is of paramount importance for improving patient outcomes and enhancing survival rates.

### Background

Photoplethysmography (PPG) is a non-invasive optical technique that measures blood volume changes in the microvascular bed of tissue. It works by shining a light source, typically an LED, onto the skin and detecting the amount of light that is transmitted or reflected back to a photodetector. This optical signal can be used to derive information about blood flow, heart rate, and other physiological parameters.

Scientific literature consistently underscores the criticality of timely recognition and intervention in cardiac arrest cases. Rapid identification of cardiac arrest triggers immediate initiation of life-saving measures, including vital procedures such as cardiopulmonary resuscitation (CPR) and defibrillation. Studies have unequivocally demonstrated that each minute of delay in commencing CPR and defibrillation reduces survival rates by 7-10%. Hence, early intervention significantly enhances the prospects of restoring normal heart rhythm, preventing irreversible damage to vital organs, and saving lives. Trained emergency response personnel and medical staff in hospitals or skilled nursing care facilities were the main users of defibrillator devices in the past, primarily when responding to a cardiac arrest incident at the location of the individual.

US 20200305737 proposes a method for monitoring heart rhythm disturbance based PPG signals wherein a heart rhythm disturbance is detected when the PPG signals drop below a predetermined threshold.

Nowadays, there are widely available automated versions of defibrillators known as “automated external defibrillators” (AEDs). An automated external defibrillator (AED) is a portable electronic device used to treat sudden cardiac arrest (SCA). It is designed to deliver an electric shock to the heart, known as defibrillation, to restore a normal heartbeat. AEDs are user-friendly and equipped with built-in instructions to guide bystanders in their use. They analyze the heart's rhythm and determine if a shock is needed. When applied promptly, typically within minutes of SCA onset, AEDs greatly increase the chances of survival. These life-saving devices are often found in public spaces, workplaces, and healthcare settings to ensure immediate access during emergencies and are designed to be used by individuals with minimal or no medical training.

Promptly alerting individuals and emergency services when a cardiac arrest occurs remains a critical need. The presence of CPR training and automated AED technology, although widely available, may go unnoticed during the crucial initial minutes in various settings such as offices, homes, or care facilities. This oversight can lead to tragic outcomes, as potential responders may not be aware of the situation. Effective notification systems are vital to ensure swift intervention. By immediately notifying family members, neighbors, office workers, and care facility staff of a cardiac arrest incident, the chances of timely life-saving interventions significantly increase, and the risk of sudden cardiac death is reduced.

### BRIEF SUMMARY OF THE INVENTION

It is an object of the current invention to correct the short-comings of the prior art and to provide a solution for accurate single-site measured PPG signals in order to detect potential cardiac arrests. This and other objects which will become apparent from the following disclosure, are provided with a light-emitting wearable device, having the features of one or more of the appended claims.

In a first aspect of the invention, the light-emitting wearable device for processing a photoplethysmography signal and detecting a cardiac arrest of a wearer, wherein the photoplethysmography signal comprises a plurality of pulses wherein a pulse is the photoplethysmography signal between two consecutive valleys, wherein the wearable device is configured for:
filtering the photoplethysmography signal by removing frequencies outside a predefined frequencies range using a bandpass filter;
calculating at least one of the following values for each pulse of the filtered photoplethysmography signal:
   a pulse wave amplitude left by calculating a difference between amplitudes of a first peak and a first valley of the pulse;
   a pulse wave amplitude right by calculating a difference between amplitudes of the first peak and a second valley of the pulse;
   a pulse wave duration by calculating a difference between a time of the second valley and a time of the first valley of the pulse;
   a rise time by calculating a difference between a time of the first peak and a time of the first valley of the pulse;
   a systolic-to-diastolic duration ratio by calculating the ratio between a difference between the rise time and a difference between the time of the second valley and the time of the first peak of the pulse;
eliminating a photoplethysmography pulse when:
   the rise time of said pulse is outside a first time range e.g. [0.01-10] seconds or [0.08-0.49] seconds; or
   the pulse wave duration of said pulse is outside a second time range e.g. [0.01-10] seconds or [0.3-2] seconds; or
   a ratio between the pulse wave amplitude right and the pulse wave amplitude left is smaller than a first threshold value e.g. 5 or 3 or 0.4; or
   systolic-to-diastolic duration ration is larger a second threshold value e.g. 0.1 or 0.5 or 1.1;
eliminating a current photoplethysmography pulse when:
   a ratio between the rise time of a preceding photoplethysmography pulse and the rise time of said current photoplethysmography pulse is larger than a third threshold value e.g. 10% or 20% or 33%; or
   a ratio between the pulse wave duration of a preceding photoplethysmography pulse and the pulse wave duration of said current photoplethysmography pulse is larger than a
   a ratio between the pulse wave amplitude of a preceding photoplethysmography pulse and the pulse wave amplitude of said current photoplethysmography pulse is larger than a fifth threshold value e.g. 10% or 20% or 50%;
calculating an average value of amplitudes of non-eliminated photoplethysmography pulses;
monitoring a plurality of consecutive pulses (e.g. 4 pulses) after detecting a photoplethysmography pulse with an amplitude lower than a fraction (e.g. 10% or 20% or 33%) of said average value;
triggering an alarm when an amplitude of said plurality of consecutive pulses is lower than said fraction of the average value.

A band-pass filter, such as second-order Butterworth band-pass filter, optionally with a frequency range of [0.2-10] Hz or [0.67-5] Hz can be applied to the PPG signals (preferably the green PPG signals) to filter out high-frequency noise and smooth the PPG pulse signals. This filtering will remove the large DC component (baseline signal) of the PPG data as well as some noises due to motion artifacts, ambient light and 50-60 Hz power line interference. What will remain after filtering is the AC component (PPG pulse signal) of the PPG.

Suitably, the wearable device of the current invention is configured for calculating an average value of amplitudes of non-eliminated photoplethysmography pulses. Said average value of amplitudes is the cardiac arrest threshold value. Once the amplitude of the peak of the PPG signal is below fraction (e.g. 33%) of the average value, a potential cardiac arrest event is detected.

After determining the threshold value for which the PPG amplitude is seen as potential cardiac arrest, it is preferable to continue monitoring the quality of the PPG signal to determine if there is still a normal PPG signal available, but with a small amplitude, or if the PPG signal is no longer detected at all. To confirm the potential cardiac arrest event the method of the current invention comprises the steps of monitoring a plurality of consecutive pulses after detecting a photoplethysmography pulse with an amplitude lower than a fraction of said average value, and triggering an alarm when an amplitude of said plurality of consecutive pulses is lower than said fraction of the average value. If a normal PPG pulse with low amplitude is detected, no alarm is given. Once a plurality (e.g. four) consecutive normal PPG pulses above the threshold are detected it is preferrable to go back to the step of monitoring the quality of the PPG signal. If below the threshold no normal PPG pulse is detected, there still is a potential cardiac arrest.

It is preferrable to only clear the potential cardiac arrest when a plurality (e.g. four) of consecutive normal pulses are detected within a first time duration (e.g. 3 seconds). The plurality of consecutive normal pulses are defined by a PPG pulse when the signal quality index is indicates a normal pulse. For instance, when for the first time duration (e.g. 3 seconds) no PPG signal is detected but then there is one normal PPG pulse and after that the PPG signal is gone again, the risk for a potential cardiac arrest will not be cleared.

Advantageously, the wearable device is configured for stopping the alarm when detecting, within the first time duration (e.g. 5 seconds), a plurality of consecutive pulses (e.g. four) having amplitudes higher than said fraction of the average value of amplitudes of non-eliminated photoplethysmography pulses.

More advantageously, the wearable device is configured for stopping the alarm when:
the wearer presses a button on the wearable device for indicating a false alarm; and
the wearer presses said button within a preset second time duration (e.g. 10 seconds).

It is convenient to alert close ones, emergency services and other third parties of the cardiac arrest event. For that end, the method conveniently comprises the steps of using GPS components of the wearable device to collect location coordinates of the wearer and sharing said location coordinates with a third party.

To save the battery life and the processing power of the wearable device, the wearable device is configured for performing the steps of the method according to the preceding operations only when a wearing detection test is positive wherein said test comprises the steps of:
registering a lowest amplitude and a highest amplitude of the filtered photoplethysmography signal during a third time duration (e.g. 6 seconds) after detecting a pulse wave with an amplitude higher than a sixth threshold value (e.g. 2× highest amplitude of the PPG within the third time duration);
calculating a fluctuation range by subtracting the lowest amplitude from the highest amplitude; and
registering a negative result to the wearing detection test when the fluctuation range is lower than a seventh threshold value.

When the wearable device is worn correctly on the wrist of the wearer, the PPG data includes small fluctuations caused by physiological signals like heart rate and blood flow. The moment someone takes off the bracelet from the wrist, a big and short jump is overserved in the PPG data due to big movements of the bracelet in the air. It is, therefore, preferable to first checks for any big jumps in the PPG data. If no jump is detected, the state of the wearing will stay the same. If a jump on the PPG data is detected, the method comprises checking for the fluctuations in the PPG signal.

Furthermore, the wearable device is configured for:
calculating a mean value of the lowest amplitudes of a plurality of wearers wearing the wearable device for a plurality of hours; and
setting the seventh threshold value at a fraction (e.g. 20%) of said mean value.

Advantageously, the wearable device is configured for performing a wearing detection test comprising the steps of:
measuring an acceleration of the wearable device movements;
registering a negative result to the wearing detection test when said acceleration is lower than an eighth threshold value during a fourth time duration (e.g. 6 seconds).

More advantageously, the wearable device is configured for:
measuring the acceleration of the wearable device movements and collecting a lowest acceleration of the wearable device movements during a fifth time duration (e.g. 5 seconds);
calculating a mean value of the lowest acceleration of the wearable device movements of a plurality of wearers; and
setting the eighth threshold value at a fraction (e.g. 20%) of said mean value.

Suitably, the wearable device is configured for of measuring an acceleration of the wearable device movements comprising the steps of:
collecting acceleration data of axis x, axis y and axis z from an accelerometer imbedded in the wearable device;
calculating a time derivative of the acceleration data of axis x, a time derivative of the acceleration data of axis y and a time derivative of the acceleration data of axis z; and
calculating the acceleration of the wearable device movement by calculating a square root of a sum of each time derivative of the acceleration data squared.

Additionally, the wearable device of the current invention is configured for registering a fall event when the acceleration of the wearable device movement is larger than a nineth threshold value (e.g. 3 g, wherein g=9.8 m/s) within a sixth time duration (e.g. 2500 ms).

Advantageously, the wearable device of the current invention is configured for:
collecting a timestamp of peak acceleration when registering the fall of the wearer within the sixth time duration;
collecting a timestamp of impact start when the acceleration of the wearable device movement is lower than a tenth threshold value (e.g. 0.8 g) within the sixth time duration (e.g. 2500 ms);
collecting a timestamp of impact end when the acceleration of the wearable device movement is larger than an eleventh threshold value (e.g. 1.5 g) within the sixth time duration; and
calculating acceleration parameters proportional to the acceleration of the wearable device movement at each time stamp or at an arithmetic combination of at least two timestamps.

More advantageously, the wearable device of the current invention is configured for training a neural network using a gradient boosting model and a training dataset comprising the calculated acceleration parameters and the registered fall events by minimizing a loss function on the training dataset.

In a second embodiment of the invention, the wearable device comprises a computer loaded with a computer program wherein said program is arranged for causing the computer to carry out the steps of the operations according to any one of aforementioned steps.

Reference to a first “aspect” of an invention is not intended to suggest that such embodiment is a preferred embodiment or best mode. Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 is a schematic diagram illustrating operations performed by a wearable device according to an embodiment of the present invention.

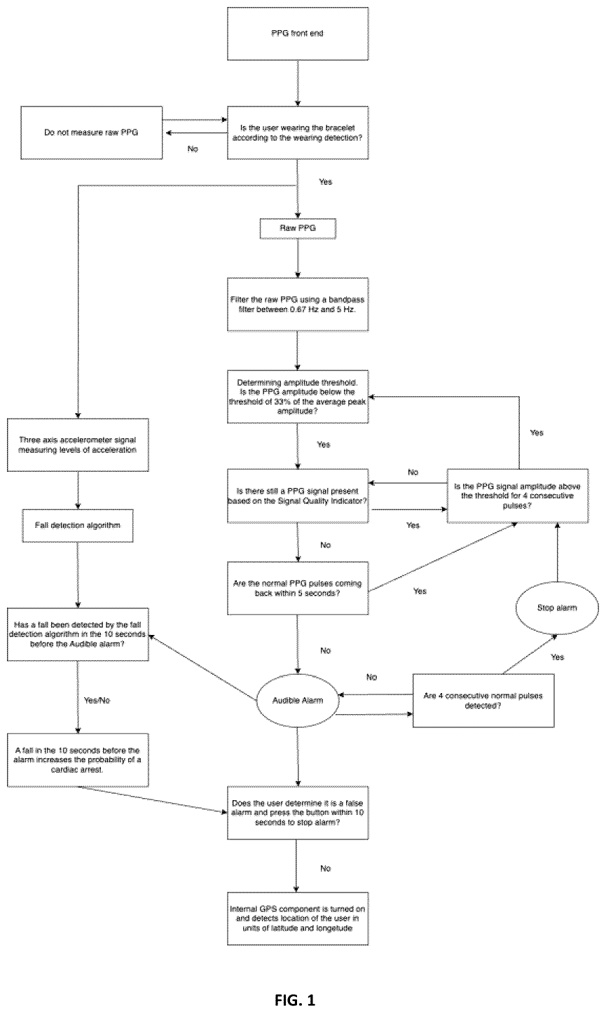

### DETAILED DESCRIPTION OF THE INVENTION

Whenever in the figures the same reference numerals are applied, these numerals refer to the same parts.

### Detailed Description

### The PPG Front End:

The PPG front end part of the method is designed to detect and measure optical signals emitted by the LED lights which is reflected back from the skin of the wearer. The LEDs under the wearable device emit light (green, red and infrared) into the user's skin, and the photodiodes capture the amount of light that is reflected back. This reflected light contains valuable information about the wearer's blood perfusion, which is the variation in blood volume in the wrist's blood vessels.

The PPG front end comprises using an analog front end (AFE) circuit to cancel out any interference caused by ambient light. Since ambient light can significantly impact the accuracy of PPG readings, the AFE helps minimizing its influence, ensuring more reliable results. Then the AFE converts the analog PPG signals into a digital format, making them easier to process and analyze. These digital PPG signals contain oscillations caused by changes in blood perfusion, providing valuable insights into the wearer's pulse rate, respiration rate, blood oxygen levels, blood pressure, etc.

The PPG front end comprises using a configurable LED driver, which allows the microcontroller of the wearable device to control the power output to the LEDs. This control enables adjustments in the intensity and timing of the LED pulses, optimizing the PPG measurements and enhancing the overall performance of the sensor system to cope with different skin colors and different wearing conditions. Every second the PPG value of every channel (i.e. color; green, red, infrared) is processed (independently from other channels). If the mean PPG value is less than an expected threshold, then the microcontroller will increase the emitting power of the corresponding LED. If the PPG value is more than a certain threshold the LED power will be decreased.

The microcontroller of the wearable device can also control the sampling frequency of the PPG signals from 25 Hz to 256 Hz depending on the requirements of the algorithms.

### Wearing Detection:

The wearing detection step of the invention comprises the step of utilizing the PPG (Photoplethysmography) and/or accelerometer (Acc) data to determine whether the wearable device is being worn by the user. Here's how it works:
PPG Data: When the wearable device is worn correctly on the wrist, the PPG data includes fluctuations caused by physiological signals like heart rate and blood flow. If the bracelet is not being worn, the PPG sensor will not receive the expected signals, and the PPG data will not show normal patterns of the pulse rate.
Accelerometer Data: The wearable device comprises an accelerometer, which is used to detects motion and orientation changes. The accelerometer can measure the acceleration forces acting on the bracelet. When a user wears the wearable device, their wrist movements and the gravitational forces acting on the device generate distinct patterns of acceleration.
Combined Analysis and algorithm: the PPG and accelerometer data can be simultaneously analyzed. This analysis determines whether the bracelet is being worn. Wearing detection algorithms use predefined thresholds and machine learning techniques to classify the combined PPG and accelerometer data. These algorithms are trained on large datasets to accurately identify the patterns associated with wearing and non-wearing scenarios.

### Band Pass Filtering:

A second-order Butterworth band-pass filter is applied to the green PPG signals to filter out high-frequency noise and smooth the PPG pulse signals. This filtering will remove the large DC component (baseline signal) of the PPG data as well as some noises due to motion artifacts, ambient light and 50-60 Hz power line interference. What will remain after filtering is the AC component (PPG pulse signal) of the PPG. This filtering process is applied to the PPG green continuously.

### Calculating Threshold Values:

The high and low peaks of filtered PPG signals are determined as the local maxima/minima points by a comparison of neighboring values. Suitably, the of the wearable device of the current invention is configured for extracting four features are for every “pulse” of PPG signal. A “pulse” is defined as one low peak (AKA valley) to the next low peak.

To assess the quality of the PPG signals, the the wearable device of the current invention is configured for calculating the following values:

$$Pulse wave amplitude left : {PWA}_{left} = {V}_{peak 1} - {V}_{valley 1} .$$

$$Pulse wave amplitude right : {PWA}_{right} = {V}_{peak 1} - {V}_{valley 2} .$$

$$Pulse wave duration : PWD = {T}_{valley 2} - {T}_{valley 1}$$

$$Rise time : RT = {T}_{peak 1} - {T}_{valley 1}$$

$$Systolic - to - diastolic duration ratio :$$

$$SDR = ( {T}_{peak 1} - {T}_{valley 1} ) / ( {T}_{valley 1} - {T}_{peak 1} ) .$$

Advantageously, the wearable device of the current invention comprises the step of eliminating the low quality ppg signals when at least one of the conditions below holds:
The PWD is outside a second time range such as [0.3-2] seconds;
The min {PWA_{right}/PWA_{left}, PWA_{left}/PWA_{right}} is smaller than a first threshold value such as 0.4.
The SDR is larger than a second threshold value such as 1.1.

In the next stage, the feature varieties of the neighbor pulses are considered. There are three conditions. If the pulse fits any condition, the quality of the pulse is annotated low and is eliminated. The three conditions are explained as following (n represents the nth pulse):
The ratio of RT(n−1) and RT(n) is larger than a third threshold value such as 33%.
The ratio of PWD (n−1) and PWD(n) is larger than a fourth threshold value such as 50%.
The ratio of PWA (n−1) and PWA(n) is larger than a fifth threshold value such as 50%.

By eliminating the low quality PPG pulses, only PPG signals with normal quality are used to determine the cardiac arrest threshold for which the start of a potential cardiac arrest is detected.

After determining the threshold value for which the PPG amplitude is seen as potential cardiac arrest, the wearable device of the current invention is configured for determining whether there is still a ppg signal available, but with a small amplitude, or whether the PPG signal is no longer detected at all.

The potential cardiac arrest is only clearer when a plurality of consecutive normal pulses (e.g. four) are detected. For instance, when for the first time duration (e.g. 3 seconds) no PPG signal is detected but then there is one normal PPG pulse and after that the PPG signal is gone again, the risk for a potential cardiac arrest will not be cleared.

If the PPG signal is no longer detected within the first time duration, an alarm is triggered. The alarm is stopped only if a plurality (e.g. four) consecutive normal PPG pulses are detected.

### Fall Detection:

The fall detection step is used as an extra check to increase the probability of the cardiac arrest. The wearer may be on their feet when the cardiac arrest occurs. Normally this leads to the user than falling as there is no cardiac output.

An accelerometer is a device that measures proper acceleration. Proper acceleration is the rate of change in velocity of a body in its own rest state. Proper acceleration contains changes in velocity for X, Y and Z axis. Accelerometer data (32 Hz) comprise data for X, Y and Z axis. The data can be processed in 2500 ms intervals (windows). For every window the total acceleration is calculated with the formula below, where t denotes a timestep in a window. total acceleration (t)=\sqrt{(Xt²+Yt²+Zt²)}

The wearable device includes an accelerometer, which can be used to detects motion and orientation changes. The accelerometer can measure the acceleration forces acting on the wearable device. When a user wears it, their wrist movements and the gravitational forces acting on the device generate distinct patterns of acceleration. To detect movements intensity, the difference acceleration vector in each axis is calculated:

$$d\_acc \_x = acc\_x ( t ) - acc\_x ( t - 1 )$$

$$d\_acc \_y = acc\_y ( t ) - acc\_y ( t - 1 )$$

$$d\_acc \_z = acc\_z ( t ) - acc\_z ( t - 1 )$$

where acc_x(t), acc_y t), acc_z(t) denote respectively the accelerometer data of axis x, y and z at time “t”,
and then the norm of the vector would be an indication of the movement intensity: N_acc=SQRT [(d_acc_x)²+(d_acc_y)²+(d_acc_z)²].
If N_acc is smaller than a threshold T_acc for 6 consecutive seconds, “no movement” (NMOV) is declared. The threshold T_acc is defined as 20% of the “average low N_acc” (ALN_acc). The ALN_acc is estimated experimentally by calculating the mean of the minimum N_acc of a population of users wearing the device day and night for 24 hours.
Combined Analysis and algorithm: The two above processes are combined and done simultaneously in one process. This process determines if the bracelet is being worn or not. If NPF and NMOV are detected at the same time, “not wearing” is declared.

### Window Shifting:

After the data is processed windows of 2500 ms can be created.

For every window the highest peak of total acceleration is selected only if there are no other peaks in the window [t-2500 ms, t]. Following that, the selected peak can be marked as peak time (pt) if the acceleration for that timestep (a_t) is larger than 3 g. This shows a peak in total acceleration has been found, which could entail a possible fall event. If this criterium is not met, the window is discarded and shifts forward.

To verify the peak, two timestamps are collected, impact end (ie) and impact start (is). These features denote the last time the total acceleration was above the eleventh threshold value (e.g. 1.5 g), and the first time the total acceleration was below a tenth threshold value (e.g. 0.8 g) respectively. In addition, (ie) should be within [t-2500 ms, t] and (is) should be within [ie-1200 ms, pt]. If (ie) is not found, then it is fixed to (ie)=pt+1000 ms.

In the peak detection step of the method, three features are obtained: pt, ie and is. These features are used to calculate the acceleration parameters that are proportional to the acceleration of the wearable device movement at each time stamp or at an arithmetic combination of at least two timestamps:

Average Absolute Acceleration Magnitude Variation, where N denotes the number of timesteps in the window:

$$AAMV = \text{?} \frac{\text{?}}{N}$$

$$\text{?} \text{indicates text missing or illegible when filed}$$

### Maximum Peak Index:

$$MPI = \text{?} ( {a}_{t} )$$

$$\text{?} \text{indicates text missing or illegible when filed}$$

### Minimum Valley Index

$$MVI = \text{?} ( {a}_{t} )$$

$$\text{?} \text{indicates text missing or illegible when filed}$$

Peak Duration Index, with ps being the start of the detected peak, defined by the last timestep where the total acceleration was lower than 1.8 g before pt. pe is denoted as the end of a peak and is the first time where the total acceleration is lower than 1.8 g after pt.

$$PDI = pe - ps$$

Activity Ratio Index, ARI, calculated as the ratio between the number of samples not in [0.85 g, 1.3 g] and the total number of timesteps in the 700-ms interval centered around (is+ie)/2.

Free Fall Index, FFI, the average magnitude in the interval [t_FFI,pt], where tFFi is denoted as the time between the first total acceleration below 0.8 g occurring up to 200 ms before pt, if not found we set this value to [pt-2200, pt].

Step Count Index, SCI, measured as the number of peaks in the interval [pt-2200 ms, pt].

Using these features, time windows can be flagged as possible fall events and stored as an event to train a prediction model in the next step of the method of the current invention.

### Model and Inference:

To train the fall detection model the wearable device of the current invention is configured for using a dataset W={w₀, w₁, . . . , wₜ}, where w; denotes a data sample (window) that consists of features xᵢ∈X and a label yᵢ∈Y, with i,t∈ [symbol] N. yᵢ yields 1 if a window is a fall event and 0 if a window is a false fall event.

| W | AAMV | IDI | MPI | MVI | PDI | ARI | FFI | SCI | Fall or no Fall |
|---|---|---|---|---|---|---|---|---|---|
| w0 | ±2.753 | 0.215 | 105 | 105 | 0.07 | ±0.314 | 1.06 | 1 | 0 |
| w1 | ±4.512 | 0.72 | 40 | 101 | 0.205 | ±0.907 | ±2.012 | 1 | 0 |
| w2 | ±1.900 | 0.54 | 99 | 179 | 0.075 | ±0.184 | ±1.009 | 0 | 1 |

Let W be w0, w0 is denoted as an event consisting of:

x0={2.753, 0.215, 105, 0.07, 0.314, 1.06, 1} y0=0

With the obtained dataset a cis trained and hypertuned. The Gradient Boosting Model combines the predictions of several weak learners to adjust the model weights of the overall model. Since, the practical application of this model should have low false positives and a high accuracy the training of the neural network optimizes for those tasks.

After the model has been trained, inference can be done on new data samples. The accuracy of the model will only increase until convergence by the Law of Large Numbers. Therefore, the model will be continuously optimized and trained with new data points obtained after deployment.

Although the invention has been discussed in the foregoing with reference to an exemplary embodiment of the device of the invention, the invention is not restricted to this particular embodiment which can be varied in many ways without departing from the invention. The discussed exemplary embodiment shall therefore not be used to construe the append-ed claims strictly in accordance therewith. On the contrary the embodiment is merely intended to explain the wording of the appended claims without intent to limit the claims to this exemplary embodiment. The scope of protection of the invention shall therefore be construed in accordance with the appended claims only, wherein a possible ambiguity in the wording of the claims shall be resolved using this exemplary embodiment.

Embodiments of the present invention can include every combination of features that are disclosed herein independently from each other. Although the invention has been described in detail with particular reference to the disclosed embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguration of their relationships with one another. The terms, “a”, “an”, “the”, and “said” mean “one or more” unless context explicitly dictates otherwise. The terms “about” or “approximately” as used herein, mean an acceptable error for an articular recited value, which depends in part on how the value is measured or determined. In certain embodiments, “about” can mean one or more standard deviations. When the antecedent term “about” is applied to a recited range or value it denotes an approximation within the deviation in the range or value known or expected in the art from the measurement method. For removal of doubt, it should be understood that any range stated in this written description that does not specifically recite the term “about” before the range or before any value within the stated range inherently includes such term to encompass the approximation within the deviation noted above.

Optionally, embodiments of the present invention can include a general or specific purpose computer or distributed system programmed with computer software implementing steps described above, which computer software may be in any appropriate computer language, including but not limited to C++, FORTRAN, ALGOL, BASIC, Java, Python, Linux, assembly language, microcode, distributed programming languages, etc. The apparatus may also include a plurality of such computers/distributed systems (e.g., connected over the Internet and/or one or more intranets) in a variety of hardware implementations. For example, data processing can be performed by an appropriately programmed microprocessor, computing cloud, Application Specific Integrated Circuit (ASIC), Field Programmable Gate Array (FPGA), or the like, in conjunction with appropriate memory, network, and bus elements. One or more processors and/or microcontrollers can operate via instructions of the computer code and the software is preferably stored on one or more tangible non-transitive memory-storage devices.

1. A light-emitting wearable device for processing a photoplethysmography signal and detecting a cardiac arrest of a wearer, wherein the photoplethysmography signal comprises a plurality of pulses wherein a pulse is the photoplethysmography signal between two consecutive valleys, and the wearable device is configured for:
calculating at least one of the following values for a pulse of the photoplethysmography signal:
a pulse wave amplitude left by calculating a difference between amplitudes of a first peak and a first valley of the pulse;
a pulse wave amplitude right by calculating a difference between amplitudes of the first peak and a second valley of the pulse;
a pulse wave duration by calculating a difference between a time of the second valley and a time of the first valley of the pulse;
a rise time by calculating a difference between a time of the first peak and a time of the first valley of the pulse;
a systolic-to-diastolic duration ratio by calculating the ratio between the rise time and a difference between the time of the second valley and the time of the first peak of the pulse;
eliminating a photoplethysmography pulse when:
the rise time of the pulse is outside a first time range; or
the pulse wave duration of the pulse is outside a second time range; or
a ratio between the pulse wave amplitude right and the pulse wave amplitude left is smaller than a first threshold value; or
the systolic-to-diastolic duration ration is larger a second threshold value;
eliminating a current photoplethysmography pulse when:
a ratio between the rise time of a preceding photoplethysmography pulse and the rise time of the current photoplethysmography pulse is larger than a third threshold value; or
a ratio between the pulse wave duration of a preceding photoplethysmography pulse and the pulse wave duration of the current photoplethysmography pulse is larger than a fourth threshold value; or
a ratio between the pulse wave amplitude of a preceding photoplethysmography pulse and the pulse wave amplitude of the current photoplethysmography pulse is larger than a fifth threshold value;
calculating an average value of amplitudes of non-eliminated photoplethysmography pulses;
monitoring a predetermined number of plural consecutive pulses after detecting a photoplethysmography pulse with an amplitude lower than a pre-determined fraction of the average value; <triggering an alarm when an amplitude of the plurality of consecutive pulses is lower than the fraction of the average value.

2. The wearable device according to claim 1, wherein the wearable device is configured for filtering the photoplethysmography signal by removing frequencies outside a predefined frequencies range using a bandpass filter, and using the filtered photoplethysmography signal in the remaining steps of the method.

3. The wearable device according to claim 1, wherein the wearable device is configured for stopping the alarm when detecting, within a preset first time duration, a plurality of consecutive pulses having amplitudes higher than the fraction of the average value.

4. The wearable device according to claim 1, wherein the wearable device is configured for stopping the alarm when:
the wearer presses a button on the wearable device for indicating a false alarm; and
the wearer presses the button within a preset second time duration.

5. The wearable device according to claim 1, wherein the wearable device is configured for using GPS components of the wearable device to collect location coordinates of the wearer and sharing the location coordinates with a third party.

6. The wearable device according to claim 1, wherein the wearable device is configured for performing the operations of claim 1 only when a result of a wearing detection test is positive.

7. The wearable device according to claim 1, wherein the wearable device is configured for:
registering a lowest amplitude and a highest amplitude of the filtered photoplethysmography signal during a third time duration after detecting a pulse wave with an amplitude higher than a sixth threshold value;
calculating a fluctuation range by subtracting the lowest amplitude from the highest amplitude; and
registering a negative result to the wearing detection test when the fluctuation range is lower than a seventh threshold value, and registering a positive result to the wearing detection test when the fluctuation range is higher than the seventh threshold value.

8. The wearable device according to claim 1, wherein the wearable device is configured for:
calculating a mean value of the lowest amplitudes of a plurality of wearers; and
setting the seventh threshold value at a fraction of the mean value.

9. The wearable device according to claim 1, wherein the wearing detection test comprises the steps of:
measuring an acceleration of the wearable device movements; and
registering a negative result to the wearing detection test when the acceleration is lower than an eighth threshold value during a fourth time duration, registering a positive result to the wearing detection test when the acceleration is higher than the eighth threshold value during the fourth time duration.

10. The wearable device according to claim 1, wherein the wearable device is configured for:
measuring the acceleration of the wearable device movements and collecting a lowest acceleration of the wearable device movements during a fifth time duration;
calculating a mean value of the lowest acceleration of the wearable device movements of a plurality of wearers; and
setting the eighth threshold value at a fraction of the mean value.

11. The wearable device according to claim 10, wherein the wearable device is configured for measuring an acceleration of the wearable device movements comprises the steps of:
collecting acceleration data of axis x, axis y and axis z from an accelerometer imbedded in the wearable device;
calculating a time derivative of the acceleration data of axis x, a time derivative of the acceleration data of axis y and a time derivative of the acceleration data of axis z;
calculating the acceleration of the wearable device movement by calculating a square root of a sum of each time derivative of the acceleration data squared.

12. The wearable device according to claim 1, wherein the wearable device is configured for registering a fall event when the acceleration of the wearable device movement is larger than a nineth threshold value within a sixth time duration.

13. The wearable device according to claim 1, wherein the wearable device is configured for:
collecting a timestamp of peak acceleration when registering the fall of the wearer within the sixth time duration;
collecting a timestamp of impact start when the acceleration of the wearable device movement is lower than a tenth threshold value within the sixth time duration;
collecting a timestamp of impact end when the acceleration of the wearable device movement is larger than an eleventh threshold value within the sixth time duration; and
calculating acceleration parameters proportional to the acceleration of the wearable device movement at each time stamp or at an arithmetic combination of at least two timestamps.

14. The wearable device according to claim 1, wherein the wearable device is configured for:
training a neural network for detecting a cardiac arrest using a gradient boosting model and a training dataset comprising the calculated acceleration parameters and the registered fall events by minimizing a loss function on the training dataset.

15. The wearable device according to claim 1 wherein the wearable device comprises a computer loaded with a computer program, and wherein the program is arranged for causing the computer to carry out the operations according to claim 1.
